(19) United States
(12) Patent Application Publication
(10) Pub. No.: US 2010/0029001 A1
(43) Pub. Date: Feb. 4, 2010

(54) ROTATING TRANSFER FOR COMPLEX PATTERNING

(76) Inventors: Xiaodon Chen, Singapore (SG); Lifeng Chi, Munster (DE); Harald Fuchs, Munster (DE); Michael Hirtz, Bremen (DE)

Correspondence Address: SYLKE LAW OFFICES, LLC, 756 N. MILWAUKEE STREET, SUITE 210, MILWAUKEE, WI, 53202, US

(21) Appl. No.: 12/515,609

(22) PCT Filed: Nov. 21, 2007
(86) PCT No.: PCT/EP2007/010090

§ 371 Date: May 20, 2009

(30) Foreign Application Priority Data
Nov. 21, 2006 (GB) 0623181.5

(51) Int. Cl. B05C 3/10; B32B 9/04; B32B 17/06; B32B 15/04; B05D 3/02; C12N 5/00

(52) U.S. Cl. 435/402; 118/402; 428/411.1; 428/446; 428/426; 428/457; 427/385.5

(57) ABSTRACT

A rotating transfer device for transfer of a film to a substrate at a gas-liquid interface, the rotating transfer device comprising a substrate holder holding the substrate, and a rotational positioning unit for rotating the substrate holder, the rotational positioning unit having a pivot to which the substrate holder is attached, whereby the pivot is angled between 0° and 60° to the plane of the interface surface. As a result, LB patterns with different dimensions and orientations that depend on the transfer velocity can be generated simultaneously.

Substrate-bound molecular gradients (chemical gradients) with a well-controlled fashion offer an in vitro model to study the biological phenomena that occur in vivo, for instance axon guidance, cell signalling and proliferation. It has been demonstrated that the chemical gradients on surfaces can influence the function and development of cells, biological recognition and interaction. Different approaches to produce chemical gradients on surfaces have been reported, for instance microfluidic systems, controlled diffusion of reactive substances, and microcontact printing.

Similarly, generating continuous gradient microstructure or nanostructure on surfaces (topographical or pattern gradients) would be interesting for cell motility and adhesion, cell mechanotransduction, and micro/nano analysis systems. However, the fabrication of structure gradients on surface is much less addressed than that of concentration gradients. The reason is that it is difficult and expensive to fabricate the continuous gradient microstructures and nanostructure on surfaces over the distances required for biological studies (at least a few hundred micrometers) based on only top-down techniques, for instance scanning probe lithography and optical lithography. This inspires the search for bottom-up techniques based on self-assembly because of their simplicity, high-yield, and ease of implementation.

### PRIOR ART

German patent DE 199 28 658 C2 describes a method for forming ordered, parallel oriented channel structures in an organic material situated on a surface of a substrate. This patent describes that during execution of the Langmuir-Blodgett technique oscillations of the sub-phase meniscus are excited by using an optimal pulling velocity for the substrate. This results in periodic wetting and de-wetting of the organic material at the substrate.

UK Patent Application GB 2 144 653 A describes a method of preparing Langmuir-Blodgett multilayers on a substrate wherein the substrate is carried on a rotatable member which is partially immersed in a liquid. In GB 2 144 653 A the film transfer occurs at a superficial surface of a tubular roller, a flexible belt or web. This results in that the linear transfer speed along the three-phase contact line (i.e. the line at which the film transfer occurs) is constant for every point of the three-phase contact line.

Korean patent KR 94-08576 describes a Langmuir trough comprising, among other things, a substrate holder having both rotational and up-and-down motion, and surface pressure gauges on the left and right sides of an immersing device. The rotation of the substrate holder occurs about the vertical axis. The purpose of the substrate holder is to obtain the accurate transfer ratio of the organic materials irrespective of positions of the sensors (presumably the surface pressure gauges).

### SUMMARY OF THE INVENTION

A simple yet novel method based on the Langmuir-Blodgett (LB) technique is described to achieve a continuous gradient mesostructure in a well-ordered fashion over large areas. A floating monolayer is transferred onto a surface of a solid substrate by rotating the substrate about an axis rather than vertically (linearly) pulling the substrate out of a fluid. As a result, LB patterns with different dimensions and orientations that depend on the transfer velocity can be generated.

A rotating transfer device for transfer of a film to a substrate at a gas-liquid interface comprises a substrate holder holding the substrate, and a rotational positioning unit for rotating the substrate holder. The rotational positioning unit has a pivot to which the substrate holder is attached, whereby the pivot is angled between 0° and 60° to the plane of the interface surface. The linear velocity at different points on the substrate depends on the distance of the points of the substrate to the axis of rotation. For transferring densely packed monolayers, such as liquid condensed (LC) phase or solid phase, rotating transfer should make no difference in monolayer structure and morphology compared with vertical transfer. However, the LB transfer process itself can be used to induce phase transitions and stripe pattern formation from a homogeneous liquid expanded (LE) monolayer. The linear velocity can be used to quantitatively control the shape and size of a variety of patterns. In this case, the rotating transfer is an efficient way to simultaneously generate patterns with different dimensions and orientations on the same substrate. Having different orientations of the film on a single substrate offers new test scenarios, for example when studying the influence of a field (such as an electric, magnetic, or gravitational field) on differently oriented cells grown on the substrate. Commonly, the transfer of the film to the surface of the substrate takes place at a water-air interface, but the interface could be between other substances as well.

For the conventional LB vertical transfer, the dipper is only able to move the substrate up or down along the Z-axis (parallel to the normal of water surface) in a linear manner. The linear velocity for all points on the surface of the substrate is same. From a geometric point of view, however, the transfer of the floating monolayer onto the surface of the substrate from the air-water interface can be realized by moving the substrate along the Z-axis (vertical transfer) or by rotating the substrate about the X-axis (FIG. 1).

A pivot used to rotate the substrate may be substantially parallel to the plane of the interface surface. In this case the pivot is angled at 0° to the interface surface. In this configuration, the film transfer typically involves no shearing (under the assumption that the plane of the substrate is perpendicular to the pivot). Hence, the global geometry of the film transfer process is maintained compared to the classical conventional LB vertical transfer. However, as pointed out above, the local film transfer speed is a function of the point on the substrate. For a geometrical analysis of this function and simulation results see below.

The rotational positioning unit may comprise a motor and a gear. The motor may be an electrical DC motor. This type of motor has a nearly constant torque characteristic making it suitable for use. A gear provides for reduction of the rotational speed of the motor to fit the desired speed range required for film transfer.

The rotating transfer device may further comprise an angular control. This angular control also comprises an angular speed control. A constant angular speed will result in a non-uniform distribution of the film due to the dependency of the local transfer speed at a given point on the substrate from the distance of that given point to the pivot. However, more complex angular speed profiles may also be considered, resulting in corresponding film distributions on the substrate.

The rotating transfer device may further comprise a linear positioning unit. In this case, the LB rotating transfer device is combined with a conventional LB vertical transfer device, resulting in more versatility of the LB rotating device.

The film may consist of DPPC, nanocrystals, or polymers. The DPPC (dipalmitoylphosphatidylcholine) produces a stripe pattern on the surface of the substrate. In practice, the DPPC is mixed with NBD (nitro-benzoxdiazole) or with alkoxyamine. Alkoxyamine allows the variation of the structure of the film across the surface of the substrate. A polymer could be for example a lipopolymer.

The substrate may be selected from the group consisting of mica, single crystalline silicon, glass, quartz, polymer and gold. The silicon may be oriented in the 100 and the 111 directions. The polymer may be for example polystyrene.

The film at the gas-liquid interface may be a Langmuir film. A Langmuir film is a one-molecule thick monolayer of amphiphilic water insoluble molecules located at the water/air interface. These Langmuir films are useful as components in many applications including, but not limited to, sensors, detectors, displays and electronic circuit components. The possibility to synthesize organic molecules with a desired structure and functionality in conjunction with the Langmuir-Blodgett deposition technique enables the production of electrically, optically and biologically active components on a nanometer scale.

The device may be used for dip coating of the substrate. This technique to obtain gradient structure is low-cost and high-throughput.

The film on the surface of the substrate may have a striped shape. In the exemplary case of DPPC, the formation of stripes is due to substrate-mediated condensation of DPPC and meniscus oscillation at the three-phase contact line.

The structure of the film may vary across the surface of the substrate. LB rotating transfer may be used to produce gradient mesostructure in a well-ordered fashion on the surface. One may expect that combining the LB rotating transfer with variations in surface pressure during transfer as well as other LB transfer induced patterns would enrich the parameter space allowing the formation of even more complex patterns. It is easy to extrapolate the LB rotating transfer method presented here to other systems, such as nanoparticles, to obtain complex nanoparticle arrays.

The substrate may be substantially flat. The substrate may furthermore be attached to the pivot in a way that the substantially flat surfaces of the substrate are substantially perpendicular to the pivot. The film transfer mainly occurs at the surfaces of the substrate that are perpendicular to both the interface and the pivot. In this manner, a flat film can be obtained as opposed to devices that result in a wound film. Furthermore, the substrate may be solid and/or rigid.

The invention also relates to a method. A rotating film transfer method for transfer of a film to a substrate at a gas-liquid interface is performed by means of a sample holder. The sample holder performs a rotational movement around an axis angled between 0° and 60° to the plane of said interface surface

The axis may be substantially parallel to the plane of said interface surface.

The sample holder additionally may perform a linear movement during said transfer.

A rotational position of said sample holder may be measured.

The film may comprise DPPC, nanocrystals, or polymers.

The substrate may be selected from the group consisting of mica, single crystalline silicon, glass, quartz, polymer and gold.

The method may comprise a further step of polymerising the film. This step may serve to preserve the film.

The method may comprise a further step of growing cells on the surface of the film. Hence, the film may be used for subsequent biological or biochemical tests.

The film at the liquid-gas interface is a Langmuir film.

The invention also relates to a product made using the method described above wherein a structure of the film varies across the surface of the substrate.

### DESCRIPTION OF THE DRAWINGS

FIG. 1 is schematic perspective view of the rotating transfer device and of its working principle.

FIG. 2 is another schematic perspective view of the rotating transfer device.

FIG. 3 is a schematic side view of the substrate and the gas-liquid interface.

FIG. 4 is a drawing showing the geometry of the rotating transfer device.

FIG. 5 is a plan view of a substrate showing the distribution of the vertical linear speed on the substrate.

FIG. 6 is a plan view of a substrate showing the distribution of the angle of the three-phase contact line.

FIG. 7 shows eight fluorescence microscopy images of different locations on a substrate.

FIG. 8 is a graph of the dependence of the angle of the three-phase contact line θ (theta) on the radius, both theoretical and experimental.

FIG. 9 is a graph of the area coverage of luminescent stripe and NBD concentration as a function of the radius.

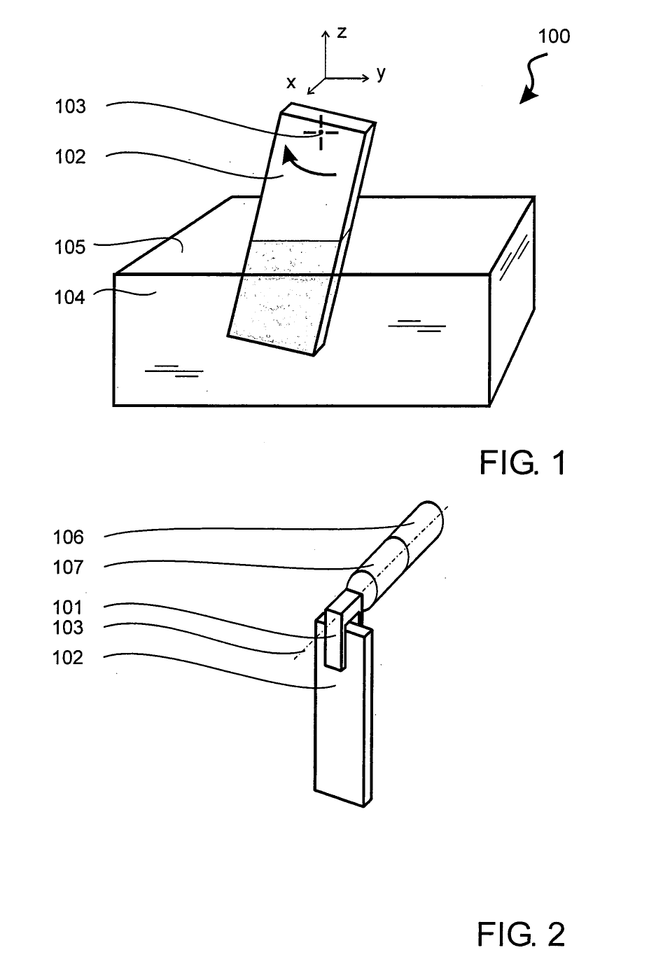

Referring to the drawings, and initially to FIG. 1, there is shown a Langmuir-Blodgett (LB) film transfer arrangement 100. The LB film transfer arrangement 100 comprises a trough 104 with a liquid inside (typically water). The liquid has an interface 105 with the air (gas) above. The arrangement further comprises a substrate 102. A pivot 103, to which the substrate 102 is directly or indirectly attached, provides rotatability to the substrate 102.

In FIG. 2, it can be seen that the substrate 102 may be attached to a frame or substrate holder 101. This allows for easy attachment and detachment of the substrate 102. In this case, the substrate 102 is indirectly attached to the pivot 103 via substrate holder 101. The LB rotating transfer device comprises further a DC motor 106, a gear 107, and an angular position control (not shown). The substrate holder 101 is used to hold the parts together and connect to a conventional Z-direction dipper. The LB rotating transfer device is connected to a standard windows PC by commercial interface which allows the computer to control the DC motor 106 and read the actual angular position of the substrate holder 101.

The position measurement is done by a potentiometer which is attached to the axis of the DC motor 106. There is a voltage drop proportional to the angular position that is digitized by the commercial interface and then used by the software to calculate the angular position of the substrate holder 101.

The software developed in Visual Basic (the programming environment was Microsoft Visual Studio 2005) is split in three parts: control-mode, setup-mode and debug-mode. In the control mode one can set the rotation speed of the substrate 102 in specified distances from the rotation axis or in rotation per minutes. The substrate holder 102 can be moved to defined positions “up” (sample parallel to the air-water surface) and “down” (sample perpendicular to the air-water surface) and the dipping process can be initiated by control buttons. In setup-mode one can chose the COM port of the PC, test the connection to the LB rotating transfer device and run the calibration to get the needed values to calculate the speed readings in control mode. In debug-mode one has direct access to the actual angular positions, and one can move to any desired position and measure the actual rotation speed as well as record graphs of the position versus time to check the status of the LB rotating transfer device.

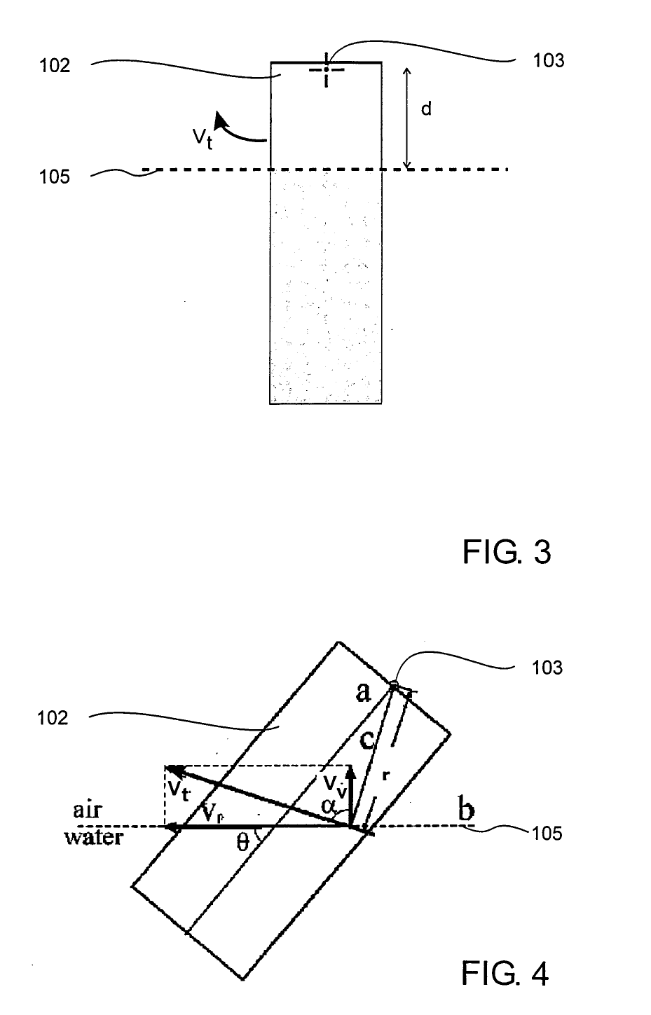

FIG. 3 shows the substrate 102 in a side view partially immersed in the liquid beneath the gas-liquid interface 105. The arrangement is shown in a typical initial state. The letter d marks the distance between the pivot 103 and the interface surface 105. The curved arrow marked vₜ illustrates the direction of rotation of the substrate, where vₜ stands for transfer velocity.

FIG. 4 illustrates the geometry of the arrangement during operation. The transfer velocity vₜ extends along the tangent of radius, i.e. it is the linear velocity at a specific point of the three phase contact line. For analysis, the transfer velocity vₜ is divided into two velocities, i.e. the velocity perpendicular to the three phase contact line (vᵥ), and the linear velocity parallel to the three phase contact line (vₚ). The geometrical relationships for these velocities are

vₜ2πωr

vᵥ=vₜ cos α

where r is the radius i.e. the distance away from the pivot 103 (axis of rotation), ω (omega) the angular velocity, α (alpha) the angle between the direction of vᵥ and vₜ, θ (THETA) the angle between line a and the three phase contact line (i.e. line b).

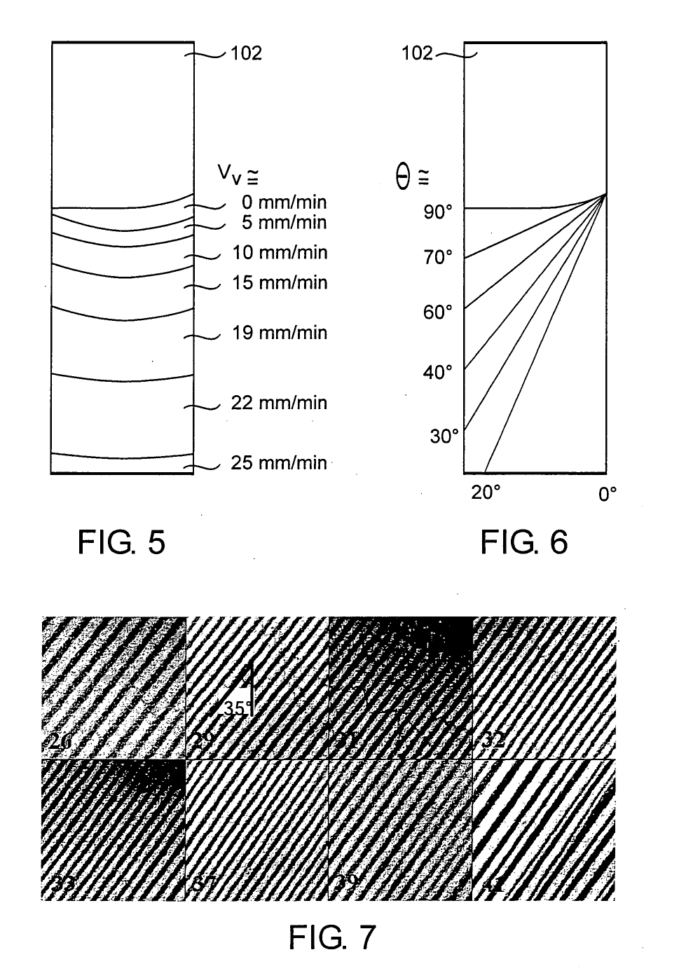

FIG. 5 shows the distribution of the velocity vᵥ on the substrate 102. As can be expected, the velocity vᵥ increases as the distance to the pivot increases.

FIG. 6 shows the distribution of the orientation stripe pattern (or value of θ (THETA)) on the substrate 102. A direct relation to the orientation of the substrate 102 at the moment when film transfer occurred at a given position on the substrate 102 can be observed.

FIG. 7 shows eight fluorescence microscopy images (30×30 μm²) for the pattern along line b at an angle θ=35° (THETA=35°). The number in the image is the radius in mm (top row from left to right: 26, 29, 31, 32; bottom row from left to right: 33, 37, 39, 41). It may be observed that the dependency of lateral width of the luminescent stripes is complicated. At first, the lateral width decreases with the radius increasing. However, at some point, the lateral width tends to increase with the radius increasing.

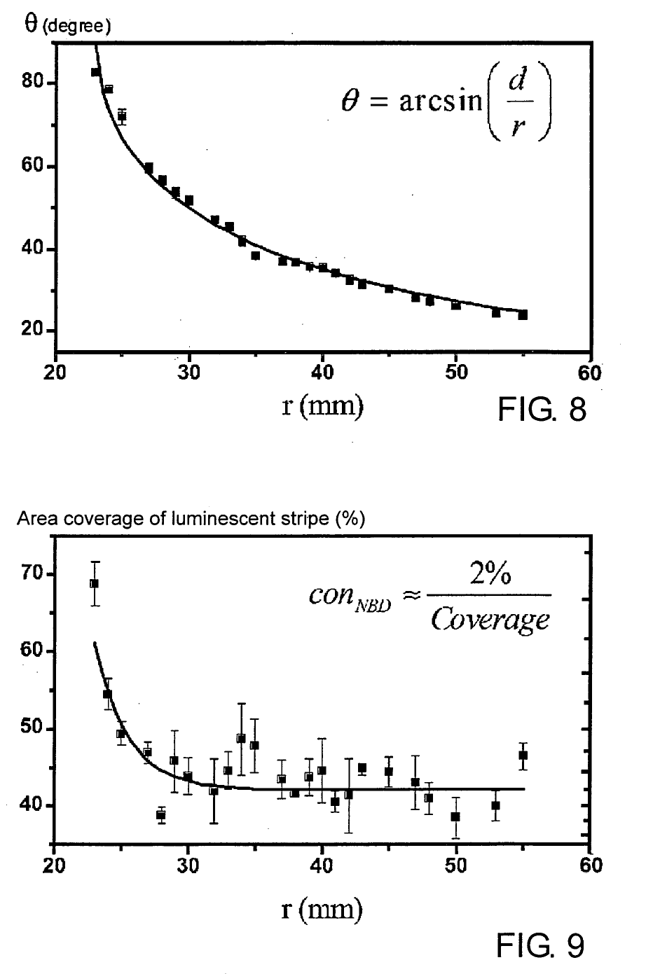

FIG. 8 shows the dependence of θ (THETA) on the radius. It can be seen that the experimental samples could be fitted well to the equation θ=arcsin (d/r) expected from geometrical considerations.

FIG. 9 shows the area coverage luminescent stripe and NBD concentration as a function of the radius. A concentration gradient can be observed. First, it is assumed that the density of DPPC in the luminescent stripes is the same as that at the air-water interface (LE phase) and the molecular size of NBD and DPPC is the same. After we have obtained the area coverage of luminescent stripes from fluorescence images, we can calculate the NBD concentration in the luminescent stripe, which shows a concentration gradient, as shown in FIG. 8. So, the method we developed here can realize chemical gradient and structural gradient simultaneously.

### EXAMPLES

A mixed monolayer of L-α-dipalmitoylphosphatidylcholine (DPPC) and 2-(12-(7-nitrobenz-2-oxa-1,3-diazol-4-yl)-amino)dodecanoyl-1-hexadecanoyl-sn-glycero-3-phosphocholine (NBD) (2 mol %) was chosen as a model system to test the feasibility of LB rotating transfer. It has been previously demonstrated that linear luminescent stripe patterns with submicrometer widths can be obtained by means of LB vertical transferring DPPC/NBD mixed monolayer onto a mica surface. The luminescent stripes were perpendicular to the transfer direction, and the width and periodicity of the luminescent stripes strongly depended on the transfer velocity. Since the linear velocity depends on the distance r to the axis of rotation for the LB rotating transfer, a gradient structure on surface of the substrate 102 was expected.

The mixed monolayer was transferred onto mica at a surface pressure of 2 mN/m with an angular velocity (ω) of 0.07 rpm. For this angular velocity, the linear velocity perpendicular to the three phase contact line (vv) can be tuned in range of 0˜30 mm/min depending on the radius (r). The obtained pattern dimensions were measured by fluorescence microscopy (Olympus BX41). However, it was difficult to measure the pattern in one image due to its length of several centimetres along the substrate 102, and the small features of pattern. Thus, two typical scenarios were selected. One scenario concerns the points along middle line a of substrate (FIG. 4). For this case, θ (the angle between line a and the three phase contact line, FIG. 4) is different for different points along line a. Another approach was to measure the points along the three phase contact line, where θ is kept constant (FIG. 7).

In summary, a simple yet novel method, LB rotating transfer, was developed to produce gradient mesostructure in a well-ordered fashion on a surface. In addition to already mentioned applications, this kind of transfer could be easily used to test the experimental conditions for exploring the pattern formation of the other systems (i.e. high-throughput studies). With the help of LB lithography and nanoimprinting, the present gradient mesostructure would serve as a platform to study cell motility and adhesion, neuron guidance, and other processes involved in biological pattern formation.

1. A rotating transfer device for transfer of a film to a substrate at a gas-liquid interface, the rotating transfer device comprising:
a substrate holder holding said substrate, and
a rotational positioning unit for rotating the substrate holder, the rotational positioning unit having a pivot to which the substrate holder is attached, and whereby the pivot is angled between 0° and 60° to the plane of the interface surface.

2. The rotating transfer device of claim 1, wherein the pivot is substantially parallel to the plane of the interface surface.

3. (canceled)

4. The rotating transfer device of claim 1, further comprising an angular control.

5. The rotating transfer device of claim 1, further comprising a linear positioning unit.

6. The rotating transfer device of claim 1, wherein said film comprises DPPC, nanocrystals or polymers.

7. The rotating transfer device of claim 1, wherein the substrate is selected from the group consisting of mica, single crystalline silicon, glass, quartz, polymer and gold.

8. The rotating transfer device of claim 1, wherein the film at the gas-liquid interface is a Langmuir film.

9. The rotating transfer device of claim 1, wherein the device is used for dip coating of the substrate.

10. The rotating transfer device of claim 1, wherein the film on the surface of the substrate has a striped shape.

11. The rotating transfer device of claim 1, wherein the structure of the film varies across the surface of the substrate.

12. The rotating transfer device of claim 1, wherein the substrate is substantially flat.

13. A rotating film transfer method for transfer of a film to a substrate at a gas-liquid interface by means of a sample holder, wherein said sample holder performs a rotational movement around an axis angled between 0° and 60° to the plane of said interface surface.

14. A rotating film transfer method of claim 13, wherein said axis is substantially parallel to the plane of said interface surface.

15. The rotating film transfer method of claim 13, wherein said sample holder additionally performs a linear movement during said transfer.

16. (canceled)

17. The rotating film transfer method of claim 13, wherein said film comprises DPPC, nanocrystals or polymers.

18. The rotating film transfer method of claim 13, wherein the substrate is selected from the group consisting of mica, single crystalline silicon, glass, quartz, polymer and gold.

19. The rotating film transfer method of claim 13, further comprising a further step of polymerising the film.

20. The rotating film transfer method of claim 13, further comprising a further step of growing cells on the surface of the film.

21. The rotating film transfer method of claim 13, wherein the film at the liquid-gas interface is a Langmuir film.

22. The rotating film transfer method of claim 13, wherein the substrate is substantially flat.

23. (canceled)
